(19) United States
(12) Patent Application Publication
(10) Pub. No.: US 2018/0085057 A1
(43) Pub. Date: Mar. 29, 2018

(54) HEALTH MONITOR SYSTEM, SENSOR, AND METHOD

(71) Applicant: INNOVAURA CORPORATION, EDMONDS, WA (US)

(72) Inventors: C. MACGILL LYNDE, BELLEVUE, WA (US); DEREK PLATT, EDMONDS, WA (US); CHRISTOPHER A. WIKLOF, EVERETT, WA (US)

(21) Appl. No.: 15/647,022

(22) Filed: Jul. 11, 2017

Related U.S. Application Data
(60) Provisional application No. 62/361,427, filed on Jul. 12, 2016.

(51) Int. Cl. A61B 5/00; A61B 5/024; A61B 5/0265; A61B 5/0255

(52) U.S. Cl. CPC A61B 5/4875; A61B 5/02444; A61B 5/7278; A61B 5/0265; A61B 2562/164; A61B 5/486; A61B 5/7282; A61B 2562/0223; A61B 5/0255

(57) ABSTRACT

A system and method can measure hydration includes using a health monitor sensor to measure pulse rate and modulation. The wearer is prompted when the pulse rate and pulse modulation exceed a limit indicative of a response to dehydration.

### CROSS REFERENCE TO RELATED APPLICATIONS

The present application claims priority benefit from U.S. Provisional Patent Application No. 62/361,427, entitled “HEALTH MONITOR SYSTEM, SENSOR, AND METHOD,” filed Jul. 12, 2016 (docket number 3012-017-02); which, to the extent not inconsistent with the disclosure herein, is incorporated by reference.

### SUMMARY

According to an embodiment, a health monitor includes a wrist-worn sensor that can detect a periodic expansion of the radial artery. The frequency of the periodic expansion is indicative of heart rate. In an embodiment, a magnitude of periodic expansion is indicative of blood volume. According to embodiments, the detected heart rate and blood volume are correlated to infer a state of hydration of the wearer. According to an embodiment, the detected heart rate and blood volume are correlated to infer a rate of caloric output.

During normal changes in hydration, human blood volume changes. Serum volume is decreased as overall hydration decreases. This can be exhibited as an overall increase in blood viscosity. Blood is a (shear-thinning) non-Newtonian fluid that is characterized by relatively high viscosity during low-shear conditions and relatively low viscosity during high-shear conditions. The systolic phase of pulse tends to be characterized by higher shear force on the blood compared to the diastolic phase. Due to blood's response to shear force, the viscosity is higher during the diastolic phase than during the systolic phase.

During exercise, moderate dehydration may be accompanied by an increase in peripheral blood pressure (BP), with diastolic BP increasing somewhat more than systolic BP, and by an increase in pulse rate. During severe dehydration, peripheral BP may decrease as the body redirects blood flow to vital organs.

A human pulse wave is characterized by a peak resulting from the heart's contraction during systole, quickly followed by a smaller peak resulting from wave reflection during diastole. The inventors have discovered that the change in systolic wave peak-to-reflected wave peak, as measured by the differential peripheral artery expansion, changes as a function of blood viscosity and can be used to estimate hydration even without a blood pressure cuff or other apparatus for measurement of absolute (or gauge) blood pressure. In an embodiment, changes in differential peripheral artery expansion may be combined with changes in pulse rate to further refine the estimate of hydration. The inventors contemplate that detected arterial expansion and heart rate may be used to infer caloric output. The inventors further contemplate detecting differential peripheral artery expansion to estimate or infer other medical, health, and/or nutritional conditions.

According to an embodiment, an increase in blood viscosity results in decreased differential expansion of peripheral arteries, with a corresponding decrease in signal modulation generated responsive to the differential expansion. The body may compensate by simultaneously increasing heart rate. In an embodiment, the health monitor sensor includes a pulse sensor that simultaneously measures heart rate and systolic peak to diastolic peak arterial expansion ratio, which is expressed as modulation. A mobile health monitor application can correlate the heart rate and modulation, estimate a hydration state of a user, and drive a user interface to alert the user to drink fluids in order to maintain optimal hydration.

Optionally, the health monitor sensor may be configured to simultaneously measure athletic exertion. For example, the health monitor sensor can measure apparent motion of far field magnetic field (e.g., earth's magnetic field) or otherwise sense accelerations corresponding to gross motor movements of the person. The mobile health monitor application can correlate the measurements to provide enhanced sensitivity and improved rejection of spurious measurements.

Optionally, the health monitor sensor can include a skin impedance sensor. Detected skin impedance combined with detected blood volume can provide data to inform a process for estimating hydration.

According to embodiments, a hydration estimation process may be performed with a programmable or application specific logic device (such as an FPGA or ASIC) or as a computational thread supported by a microcontroller or microprocessor. In an embodiment, the process may be disposed at least partly on a networked server operatively coupled to the local health monitor sensor hardware.

According to an embodiment, a computer method for monitoring the hydration of a person includes measuring a physical periodic expansion of a peripheral artery with a sensor, each measured physical periodic motion including a modulation and a pulse rate, receiving the modulation and pulse rate with a microcontroller, and saving the modulation and pulse rate to a buffer memory as a modulation history and pulse rate history. The microcontroller calculates a combined modulation and pulse rate limit from the modulation and pulse rate history. The computer method also includes writing the combined modulation and pulse rate limit to a non-transitory computer readable medium; and subsequently comparing, with the microcontroller, one or more measured instances of the modulation and corresponding pulse rate to the modulation and pulse rate limit. The microcontroller outputs a prompt via a user interface to the person if a predetermined number of measured instances of the modulation and pulse rate falls outside the modulation and pulse rate limit, indicating a probable need for rehydration. Optionally, the modulation and/or pulse rate limit may be expressed as a derivative, such that the method looks for changes in slope of modulation and/or pulse rate vs. time.

According to an embodiment, a non-transitory computer readable medium carrying computer executable instructions configured to cause a portable device to execute the method including the steps of measuring a physical periodic motion of a peripheral artery, each measured physical periodic motion including a modulation and a pulse rate; receiving the modulation and pulse rate with a microcontroller; and saving the modulation and pulse rate to a buffer memory as a modulation history and pulse rate history. The microcontroller can determine a combined modulation and pulse rate limit from the modulation and pulse rate history. The combined modulation and pulse rate limit can be written to a non-transitory computer readable medium. The microcontroller compares one or more measured instances of the modulation and corresponding pulse rate to the modulation and pulse rate limit. The microcontroller outputs, via a user interface, a prompt to the person if the one or more measured instances of the modulation and pulse rate fall outside the modulation and pulse rate limit.

The method for monitoring a human pulse can include using one or more magnetic sensor(s) to measure the change in magnetic flux arising from the perturbation of a magnetic field where such field is created by magnets or magnetic particles affixed to or embedded in an elastomeric membrane positioned on the wrist at the radial artery.

According to an embodiment, a method can extend the functionality of the apparatus to monitor relative blood flow and, along with other inputs, allows an estimate of relative state of hydration. Blood flows through arteries as waves created by the pumping action of the heart. The change in magnetic flux is proportional to the change in the radius of the artery created by the pulse wave. The method can include calculating positive changes in arterial radius during a pulse wave by a formula relating change in magnetic flux to change in radius. The formula can be derived empirically and depends on location of sensors relative to magnetic field, among other factors. The method can also include sampling the magnetic flux frequently in order to sum the radius measurements to calculate an estimate of the volume of the portion of the wave that is distending the artery during systolic and diastolic phases and calculating an index of blood flow as a function of the above wave volume multiplied by the frequency of waves (i.e. pulse rate).

Changes in hydration can result in changes in modulation, pulse and blood flow, but these changes can also be moderated by changes in exercise and body temperature.

The health monitor sensor can include a temperature sensor to measure skin temperature as an index of body temperature and accelerometer or accelerometer/gyro to monitor motion as an index of exercise. An index of hydration can be calculated as a function using the modulation, blood flow index, pulse rate, optionally body temperature index and optionally exercise index.

### BRIEF DESCRIPTION OF THE DRAWINGS

FIG. 1A is a diagram of a pulse sensor during a portion of a user's heartbeat after a pulse wave has passed and the artery has contracted, according to an embodiment.

FIG. 1B is a diagram of a portion of the pulse sensor of FIG. 1A during a systolic portion of the user's heartbeat, according to an embodiment.

FIG. 2 is a diagram of a pulse wave corresponding to data generated by a prototype pulse sensor described in conjunction with FIGS. 1A and 1B.

FIG. 3 is a flow chart of a method for detecting a heart rate, according to an embodiment.

FIG. 4 is a flow chart of a method for tracking the heart rate of a person, according to an embodiment.

FIG. 5 is a flow chart of a method for monitoring the hydration of a person, according to an embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The terms heart rate and pulse rate are used interchangeably herein.

A health monitor sensor, unless context dictates otherwise, includes a sensor that is capable of detecting a signal at a peripheral artery proportional to instantaneous blood flow and of outputting data indicative of a plurality of artery expanded states (e.g. instantaneous cross-sectional areas, instantaneous diameters, or the like) similarly to a skilled person detecting a pulse at the location(s). Embodiments described herein make use of a modulation sensitive pulse sensor. Modulation sensitive pulse sensors may include strain or pressure sensors, ultrasound transceiver sensors, photoplethysmography transceiver sensors, or magnetic sensors, as further described herein.

Embodiments of the health monitor sensor described herein include a pulse sensor that has a first portion held conformal to pulsations of the artery expressed as movements of the skin of the wearer. A second portion of the pulse sensor is configured to measure periodic displacement of the first portion relative to the wearer's body.

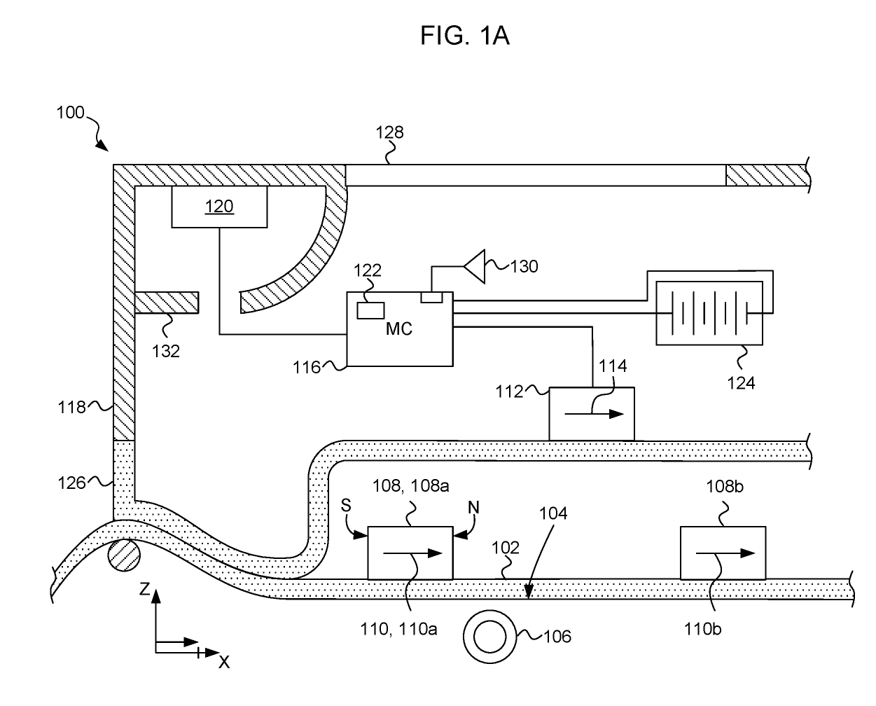

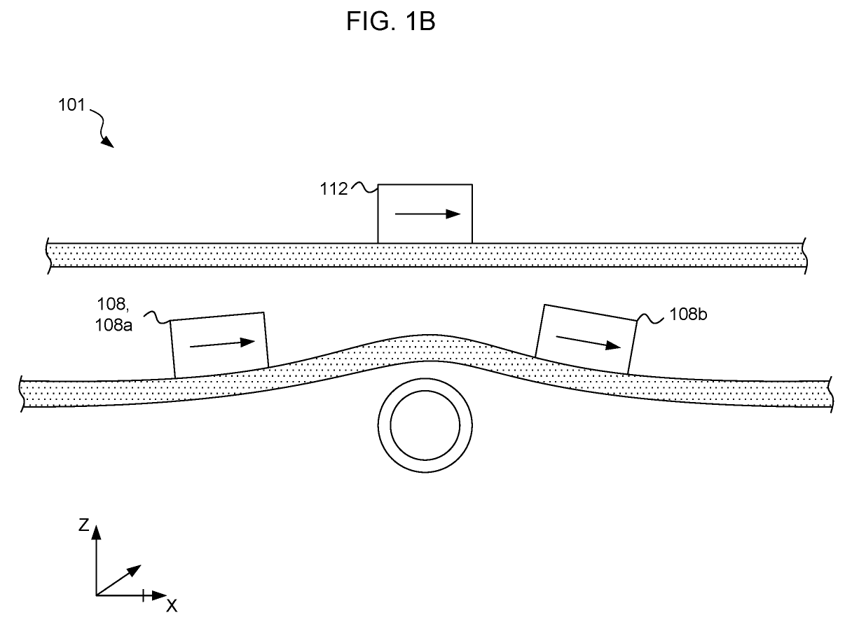

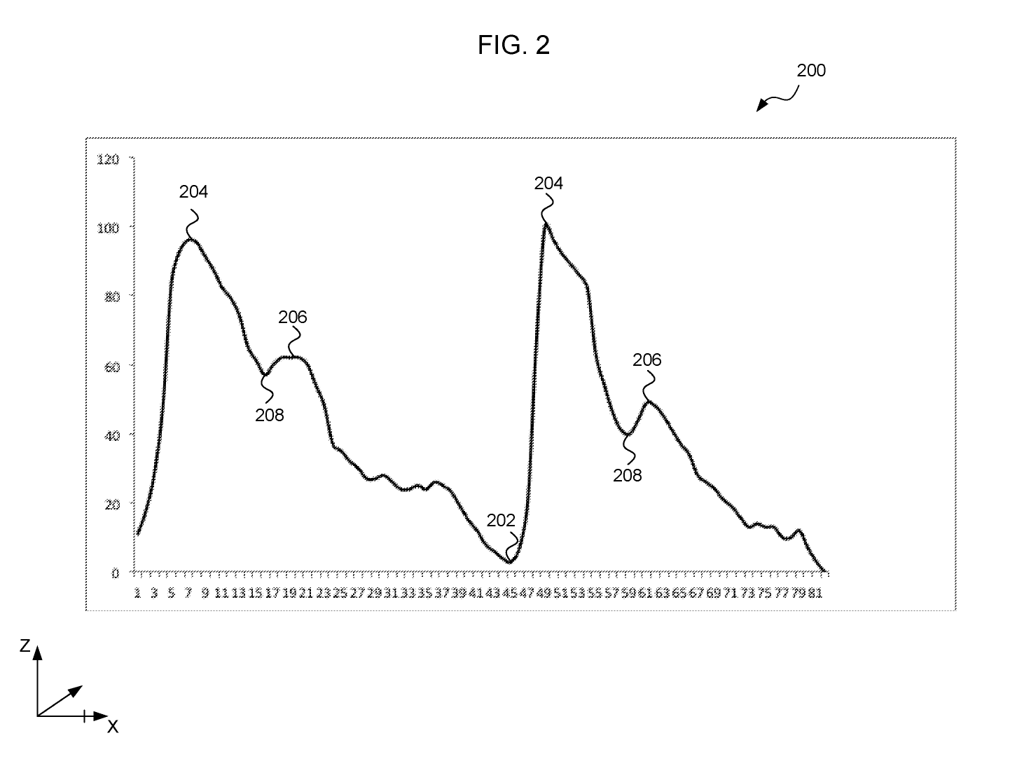

FIG. 1A is a diagram of a heart rate monitor 100, according to an embodiment. FIG. 1B is a diagram 101 of a portion of the heart rate monitor 100 of FIG. 1A during a systolic portion of the user's heartbeat after the pulse wave has passed, according to an embodiment. FIG. 2 is a diagram of a pulse wave 200 corresponding to data generated by a prototype pulse sensor described in conjunction with FIGS. 1A and 1B.

Referring to FIGS. 1A, 1B, and 2, the heart rate monitor 100 includes a flexible membrane 102 configured to be held adjacent to a user's skin 104 at a location corresponding to an artery 106 subject to pulse movement. At least one magnet 108 having a magnetic axis 110 is disposed on the flexible membrane 102. By supporting the flexible membrane 102 and the at least one magnet 108 against the user's skin 104 over the artery 106, the at least one magnet 108 can be configured to physically tilt responsive to the pulse movement, whereby the magnetic axis 110 tilts. Referring to FIG. 2, FIG. 1A corresponds to a portion 202 of a pulse wave 200 between heartbeats when the artery 106 is contracted. The magnet(s) 108 tend to lie in plane with the user's skin 104. FIG. 1B corresponds to a systole portion 204 of the heartbeat when the artery 106 is expanded under systolic pumping pressure from the heart. As can be seen, the magnet(s) 108 and corresponding magnetic axis 110 (axes) is (are) tilted up responsive to the pulse movement.

The inventors have discovered that a ratio between the (vertical axis) value of the systolic pressure 204 to the value of the diastolic hump 206 is indicative of the state of hydration of a wearer of the sensor.

As used herein, the term magnetic axis 110 is defined relative to a magnet 108; that includes a north pole (indicated as N) and a south pole (indicated as S); such that the magnetic axis 110 is a line intersecting both the north pole and the south pole of the magnet 108.

A magnetic sensor 112 is configured to measure a magnetic field produced by the at least one magnet 108, the magnetic sensor 112 having a magnetic field measurement axis 114 along which the magnetic axis tilt causes a change in measured magnetic field strength. The detected magnetic field strength varies according to the tilt angle of the magnetic axis 110 relative to the measurement axis 114. A periodicity corresponding to the detected magnetic field strength corresponds to the systolic-diastolic rhythm, and thus serves as a measurement of heart rate.

Moreover, it can be appreciated that the difference between magnet(s) angles, expressed as a difference in maximum and minimum detected magnetic field strength, can be proportional to a difference between systolic and minimum blood pressure, which can, it is contemplated, be related to gauge blood pressure of the user.

The arrangement depicted in FIGS. 1A and 1B can be especially useful for cases where the magnetic sensor 112 either does not have the ability to measure changes in magnetic field strength in the z-axis normal to the skin 104 of the user; or where the signal to noise ratio, sensitivity, or accuracy of z-axis measurement is inferior to measurements taken along the x-axis, which is nominally parallel to the magnetic axis (axes) 110 of the magnet(s) 108. This aspect can be leveraged to minimize or reduce z-axis height of the magnetic sensor 112 and/or to minimize or reduce z-axis height of a housing 118 (such as a portion of a smart watch band) that forms a portion of the heart rate monitor 100.

In some embodiments, the magnetic field measurement axis 114 can be selected to be momentarily parallel to the plane of the magnetic axis 110 of the at least one magnet 108 during a pulse period. This can occur once per period if the magnetic axis 110 is parallel to the measurement axis 114 either at diastole or at systole; or it can occur twice per period if the magnetic axis 110 is momentarily parallel to the magnetic field measurement axis 114 at a point other than maximum or minimum angular displacement (e.g., at a point in the period other than diastole or systole). In other embodiments (e.g., if the magnet 108 is at a different angular position along a curved skin surface 104 from the magnetic sensor 112), the magnetic axis 110 is never parallel to the magnetic field measurement axis 114 during heart rate measurement. Nevertheless, the measured magnetic field strength along the magnetic field measurement axis 114 will vary during the pulse period if the magnet(s) 108 is supported sufficiently close to the artery 106 that the magnet 108 tilts responsive to pulse.

As illustrated in FIGS. 1A and 1B, the at least one magnet 108 can include a plurality of magnets 108a, 108b disposed to cause at least two of the plurality of magnets 108a, 108b to physically tilt responsive to the pulse movement of the artery 106 and the skin 104. In some embodiments, there may be precisely two magnets 108a, 108b that are configured to align with the pulse point when the user dons the apparatus 100. In other embodiments, there may be three, four, or a large plurality of magnets 108a, 108b located along the flexible membrane 102, such that a gap between two of the magnets 108a, 108b will span the pulse measurement position over the artery 106. This can be used, for example, to improve tolerance for rotational displacement of the apparatus 100 around the user's wrist, improve tolerance to physical morphology differences between users, and/or allow for a looser fit of the flexible membrane 102. In yet other embodiments, a number of magnets 108 may be disposed to respond to pulse movement at a plurality of locations along a peripheral artery 106.

Referring especially to FIGS. 2A and 2B, the heart rate monitor 100 can include a microcontroller 116 operatively coupled to the magnetic sensor 112. A housing 118 can be configured to support the magnetic sensor 112 and configured to urge the flexible membrane 102 against the user's skin 104. The housing 118 can include a magnetically transparent housing portion 126 selected to pass the magnetic field produced by the magnet(s) 108 to the magnetic sensor 112. For example, the magnetically transparent housing portion 126 can be formed from a non-ferromagnetic material such as a plastic or aluminum. Additionally or alternatively, the housing 118 can be configured to support the magnetic sensor 112 between the housing 118 and the magnet 108 (configuration not shown). In this configuration, it can still be preferable for the housing 118 to be non-ferromagnetic in order to avoid distorting magnetic field lines from the magnet(s) 108.

The heart rate monitor 100 can further include a battery 124 contained within the housing 118 and configured to provide sufficient power to maintain function of the pulse sensor 100 for at least 24 hours. In some embodiments, the microcontroller 116 can go to sleep and receive motion and/or heart rate data responsive to a predetermined interval. When motion and/or heart rate is relatively constant or has a low value, the microcontroller 116 can be programmed to go back to sleep. When motion and/or heart rate data has changed since a previous sample, the microcontroller 116 can be programmed to wake up and track heart rate and motion, and output data corresponding to heart rate and motion. When motion decreases and heart rate drops, the microcontroller 116 can be programmed to go back to sleep. The combination of a low power microcontroller 116 and the inherently low power consumption of the magnetic sensor 112 used for heart rate detection can enable the battery 124 to provide sufficient power to maintain function of the pulse sensor 100 for at least one week. This is possible with current battery technology owing to the very low power consumption of the magnetic sensor 112 compared to an optical pulse sensor.

The heart rate monitor 100 can further include a motion sensor 120 operatively coupled to the microcontroller 116. For example, the motion sensor 120 can include an accelerometer or a second magnetic sensor configured to sense an ambient magnetic field that is substantially stationary relative to movements of the user. In the “second magnetic sensor” embodiment, movement of the user through the earth's magnetic field and/or other ambient magnetic fields is sensed. In the second magnetic sensor embodiment, the heart rate sensor can further include a magnetic shield 132 configured to shield the second magnetic sensor 120 from changes in magnetic field strength corresponding to movement of the magnet 108.

In another embodiment the motion sensor 120 can be integral with the magnetic sensor 112. For example, the magnetic sensor 112 can sense magnetic fields (e.g., the earth's magnetic field) along a magnetic sensor axis that is transverse to the magnetic axis 110 (e.g., along the y-axis into the plane of the drawing FIGS. 1A and 1B). This approach may result in partial confounding of movement with the pulse motion of the magnet(s) 108, but can be useful for cost reduction. In another embodiment, the motion sensor 120 can be a portion of the health monitor sensor separate from the pulse sensor. For example, the motion sensor 120 can consist essentially of an accelerometer.

The heart rate sensor 100 can further include a non-transitory computer-readable medium 122 contained within the microcontroller 116 or separate from the microcontroller 116 and operatively coupled to the microcontroller 116. In an embodiment, the non-transitory computer-readable medium 122 carries microcontroller instructions configured to cause the microcontroller 116 to receive data or a signal from the magnetic sensor 112, receive detected movement information from the motion sensor 120, and filter the data or signal from the first magnetic sensor 112 responsive to the detected movement.

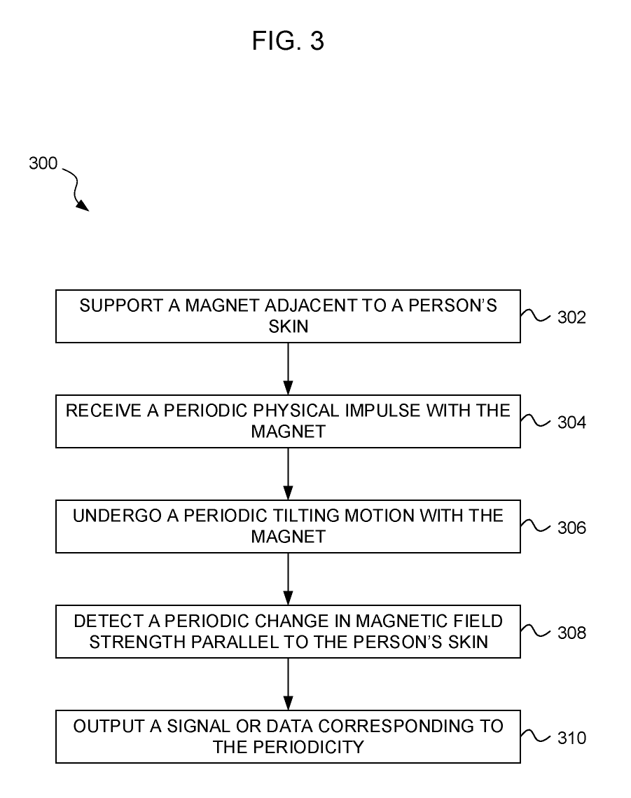

The filtering is described more fully in conjunction with FIG. 3 below.

The heart rate monitor 100 can further include an electronic display 128 operatively coupled to the microcontroller 116. The microcontroller 116 can be configured to calculate a most likely pulse rate and to cause the electronic display 128 to display the most likely pulse rate.

The heart rate monitor 100 can further include a radio 130 operatively coupled to or contained at least partially within the microcontroller 116. The microcontroller 116 can be configured to calculate a most likely pulse rate and to cause the radio 130 to transmit the most likely pulse rate, for example to a smart phone (not shown) running a fitness application that tracks the pulse rate.

Still referring to FIGS. 1A and 1B, according to an embodiment, the heart rate monitor 100 includes the flexible membrane 102 configured to be held adjacent to the user's skin 104 at a location corresponding to the artery 106 subject to pulse movement, at least one magnet 108 disposed on the flexible membrane 102 and configured to move responsive to the pulse movement, and a magnetic sensor 112 configured to measure variations in a magnetic field from the at least one magnet responsive to the pulse movement.

Other embodiments include positioning the magnetic axis 110 in a different orientation relative to the user's skin surface 104 than what is depicted in FIGS. 1A and 1B. For example, the magnet(s) can be disposed to have a vertical magnetic axis, such that the magnetic axis is substantially normal transverse to the user's skin surface 104 (e.g. up to substantially perpendicular to the user's skin surface 104), and the magnetic sensor 112 can be configured to have the measurement axis 114 that measures variations in magnetic field strength along a vertical axis substantially parallel to the magnetic axis 110. Although advantages corresponding to overcoming z-axis precision, signal-to-noise, or size may be lost, such a (normal) alignment of magnetic axis 110 and magnetic measurement axis was found by the inventors to work.

The motion sensor 120 is configured to detect movement of the human. The inventors have found that detected movement can provide data for inferring a change in heart rate. For example, an increased amount of movement may typically correspond to an increase in heart rate, and conversely a decreased amount of movement may typically correspond to a decrease in heart rate. The predictive nature of movement can be used to select from between several frequency candidates in successive signals from the magnetic sensor 112, any of which may correspond to the true heart rate.

The microcontroller 116 operatively coupled to the magnetic sensor 112 and the motion sensor 120 can include the non-transitory computer-readable medium 122 carrying microcontroller instructions. The instructions can be selected to cause the microcontroller 116 to receive data or a signal from the magnetic sensor 112, receive detected movement information from the motion sensor 120, filter the data or signal from the first magnetic sensor 112 responsive to the detected movement, and output heart rate data corresponding to the filtered data or signal from the first magnetic sensor 112.

An approach to filtering is described in greater detail below.

According to an embodiment, the heart rate sensor 100 can include sensors other than magnetic sensors for sensing the pulse rate, modulation, or blood flow rate in a peripheral artery. For example, the heart rate sensor 100 can include one or more of piezo-electric sensors, piezo-resistive sensors, capacitive sensors, or other kinds of sensors suitable for detecting parameters of a peripheral artery. Those of skill in the art will recognize, in light of the present disclosure, that sensors other than those described herein can be used in accordance with principles of the present disclosure. All such other sensors fall within the scope of the present disclosure.

FIG. 3 is a flow chart of a method 300 for detecting a heart rate, according to an embodiment. According to the method 300, a magnet is supported adjacent to the skin of a person in step 302. In step 304, a periodic physical impulse is received by the magnet responsive to arterial movement during systole and diastole. Proceeding to step 306, the magnet undergoes a periodic tilting motion responsive to the periodic physical impulse corresponding to systole and diastole. In step 308, a magnetic sensor detects, along an axis parallel to the person's skin, a periodic change in the strength of the magnetic field produced by the magnet. In step 310, a signal or data corresponding to a periodicity of the detected periodic change in the strength of the magnetic field is output. The output signal or data can correspond to a heart rate of the person.

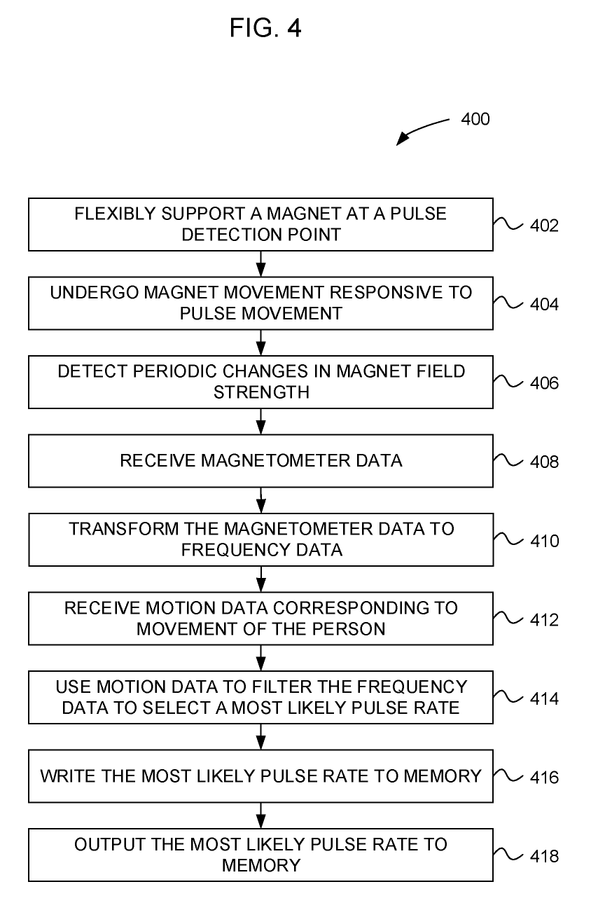

FIG. 4 is a flow chart of a method 400 for tracking the heart rate of a person, according to an embodiment. In step 402 a magnet is flexibly supported adjacent to a pulse detection location of a person. According to an embodiment, flexibly supporting a magnet adjacent to a pulse detection location of a person includes supporting a flexible membrane adjacent to the pulse detection location and supporting the magnet with the flexible membrane. For example, the flexible membrane can support the magnet adjacent to a pulse detection location on a wrist of the person. The inventors contemplate several alternative pulse measurement locations. In other examples, the flexible membrane can support the magnet adjacent to a pulse detection location on a foot or ankle of the person, adjacent to a pulse detection point on the neck of the person, or adjacent to the temple of the person.

Proceeding to step 404, the magnet undergoes movement responsive to pulse movement of the person. As described above, the movement is responsive to expansion and contraction of an adjacent artery, and especially a peripheral artery, respectively corresponding to systolic and diastolic pressure pulses from the heart. As described above, several modes of movement and detection are contemplated. In a preferred embodiment, the magnet tilts responsive to arterial pulsing, and corresponding magnetic field strength is detected along an axis substantially parallel to the skin surface of the person.

In step 406, a magnetic sensor is operated to detect periodic changes in magnetic field strength from the magnet, the periodic changes in magnetic field strength corresponding to the movement of the magnet and the pulse movement of the person.

Proceeding to step 408, a microcontroller receives magnetic sensor data including the periodic changes in magnetic field strength from the magnet. The microcontroller can, as shown in step 410, transform the magnetic sensor data to produce frequency data. For example, transforming the frequency data can include performing a Fourier transform such as a Fast Fourier Transform (FFT).

In step 412 the microcontroller receives motion data corresponding to movement of the person. The motion data can be produced by an accelerometer or another motion sensing device. In one example, the motion sensing device can include another magnetic sensor or another axis of the pulse-sensing magnetic sensor, wherein the motion data corresponds to motion of the person relative to far field sources, such as the earth's magnetic field.

In step 414 the motion data is used to filter the frequency data to select a frequency most likely to correspond to a pulse rate of the person. For example, using the motion data to filter the frequency data can include writing the frequency data to memory, writing the motion data to memory, comparing the motion data to previous motion data, determining the likelihood of a change in pulse rate responsive to the compared motion data, comparing the frequency data to previous frequency data, and identifying a high magnitude frequency domain point most likely to correspond to the pulse rate.

The method 400 can further include step 416, wherein the most likely pulse rate is written to memory; and step 418, wherein the most likely pulse rate is output. For example, step 418 can include wirelessly transmitting the most likely pulse rate to a personal electronic device. The personal electronic device can be configured to run a fitness or health application that uses the pulse rate. Additionally, or alternatively, outputting the most likely pulse rate can include displaying the most likely pulse rate on an electronic display.

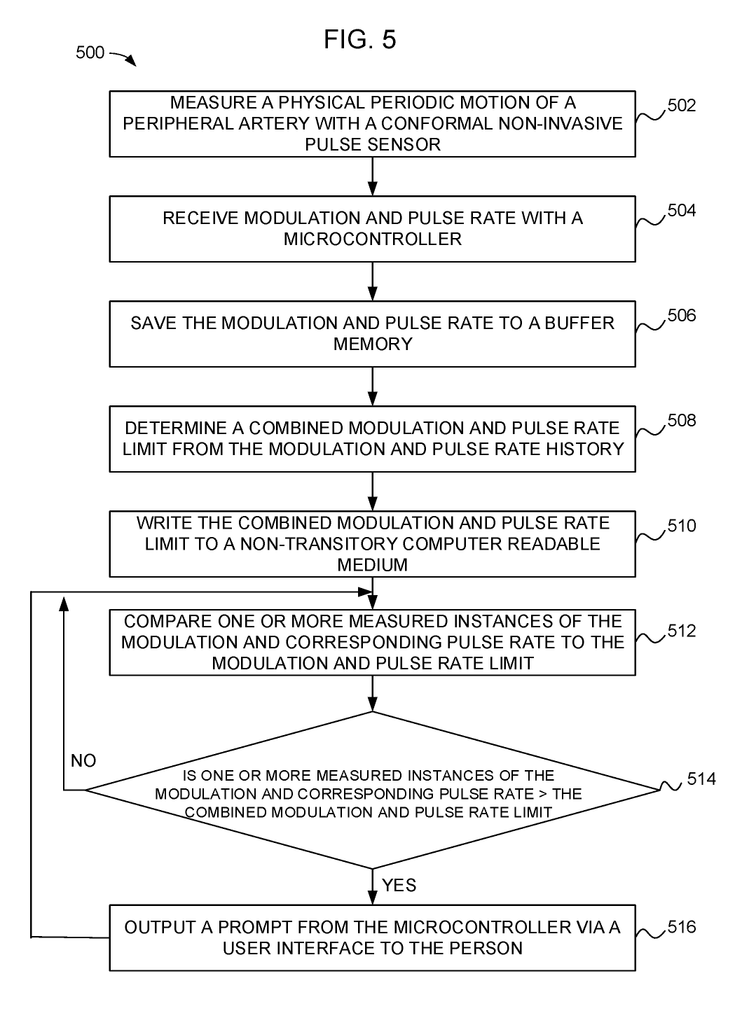

FIG. 5 is a flow chart of a method 500 for monitoring the hydration of a person, according to an embodiment. The method 500 begins at step 502, which includes measuring a physical periodic motion of a peripheral artery with a pulse sensor, each measured physical periodic motion including a modulation and a pulse rate. For example, the measurements can be performed by a heart rate monitor akin to the heart rate monitor described in conjunction with FIGS. 1A and 1B. According to an embodiment, each instance of modulation and pulse rate data can be obtained from a series of measurements of a magnetic field strength, e.g. as the field strength produced by one or more magnets that are displaced by the physical pulsations of the artery. The field strength is measured at a frequency that is a sufficiently small fraction of a pulse period to produce a waveform corresponding to changing diameters of the artery during the pulse period, and for sufficient duration to average across a plurality of periods.

Referring to FIG. 2, the modulation data for each pulse include the sequence of magnetic field strengths over the duration of the pulse period as it passes the sensor as a wave. The differences (or average difference) between maximum and minimum magnetic field strengths are used to define the magnitude of systolic expansion 204 across the periodic response of the artery (optionally, averaged along the plurality of periods). The shape and magnitude of the diastolic hump 206 is included in the modulation data, the value being determined by finding the local maximum between a local minimum 208 (dicrotic notch) following the systolic peak 204 and the overall minimum 202.

The inventors have discovered that the ratio of systolic maximum 204 to diastolic hump maximum 206 is covariant with hydration, at least over a reasonable, healthy hydration range. This relationship is used, according to embodiments, to infer a state of hydration of the measured individual.

In an embodiment, the method includes detecting periodic pulsations at a plurality of distances along an artery.

Referring again to FIG. 5, proceeding to step 504, the modulation and pulse rate are received with a microcontroller. In step 506, the microcontroller saves the modulation and pulse rate to a buffer memory as a modulation history and pulse rate history. Optionally, the microcontroller can calculate a function of the combined modulation and pulse rate (as described below) for each instance of the modulation and pulse rate pair. In this case, separately saving the actual modulation and actual pulse rate may be omitted. It will be understood that such a calculation and omission falls within the definition of “saving the modulation and pulse rate into a buffer memory.” The steps 502 to 506 can, in an embodiment, be performed asynchronously with other steps of the method 500.

In step 508, the microcontroller can determine a modulation limit, a pulse rate limit, and/or a combined limit from the modulation and pulse rate history. Optionally, step 508 can include determining separate limits for pulse rate and modulated difference between systolic peak and diastolic hump. (Under a condition of dehydration, the pulse rate increases and the difference in height between the systolic peak and diastolic hump decreases.) In another embodiment, step 508 can include determining an overall blood flow by integrating or summing the total area under the pulse wave curve over a plurality of periods, referred to as blood flow herein. (Under a condition of dehydration, pulse rate increases but blood flow decreases.) In another embodiment, step 508 can include determining both the modulated difference between systolic peak and diastolic hump and blood flow. The method may include determination of a function (that may be embodied as a look-up table, or LUT) that carries both a combined variable limit and separate single variable limits. For ease of reference, any combination of single variable and multiple variable limits may be referred to as limits, herein.

In step 510, the limits are written to a non-transitory computer readable medium. For example, the buffer memory can form a portion of the non-transitory computer readable medium.

Periodically (and optionally asynchronously with the pulse and modulation pair periodicity), referring to step 512, the microcontroller can compare one or more measured instances of the modulation, pulse rate, and/or blood flow to the corresponding limits. Step 514 is a decision step, wherein if the variables are within limits, the method 500 can loop back to step 512. If the variables are not within limits, the method proceeds to step 516. In step 516, the microcontroller outputs a prompt via a user interface to the person. Step 516 is executed only if the one or more measured instances of the variables fall outside the limits.

Outputting the prompt can take various forms. In one example, the apparatus includes a visual display such as an LED that normally pulses green approximately synchronously with the person's pulse. When the limits are violated, the microcontroller can cause the LED to pulse amber. Optionally, the variables can include different levels of limits. A more severe violation of the limits can cause the microcontroller to cause the LED to flash red. Other types of visual, audible, and haptic user interfaces for issuing the prompt may equivalently fall within meaning of “prompt.”

Various related embodiments are contemplated by the inventors.

In one embodiment, comparing the one or more measured instances of the variables to the limits in step 512 includes comparing at least three successive measured variable instances to the limits. Similarly outputting a prompt from the microcontroller via a user interface to the person if the one or more measured instances of the motion amplitude and pulse rate falls outside the limits can include outputting the prompt if and only if each of the at least three successive measured instances falls outside the limits.

As indicated above, measuring a physical periodic motion of a peripheral artery with a health monitor sensor in step 502 can include supporting one or more magnets on a flexible substrate adjacent to the person's skin such that the peripheral artery lies subjacent to the one or more magnets; and detecting a magnetic field variation produced by the one or more magnets responsive to physical pulsations received from the subjacent peripheral artery with a magnetic sensor.

The inventors contemplate a variety of approaches to determining the limits. For example, step 508 can include computing a standard deviation variable as a function of instances of variables divided by corresponding pulse rate (or the inverse thereof) and setting the limits as two standard deviations greater than a mean value. Additionally or alternatively, step 408 can include computing a slope variable of a function of successive instances of variable determination (or the inverse thereof) (e.g., a rate of change in pulse, a rate of change of modulated difference between systolic peaks and diastolic humps, and/or a rate of change in blood flow) and setting a limit as a derivative of the slope variable times a constant greater than one.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. A method for monitoring the hydration of a person, comprising:
measuring a physical periodic motion corresponding to a peripheral artery with a pulse sensor to determine pulse data based on a magnetic sensor, each measured physical periodic motion corresponding to a sequence of instantaneous arterial size estimates or derivatives thereof;
outputting the pulse data;
receiving one or more instance of pulse data with a programmable hardware device;
writing the pulse data to a memory device as an instant in an arterial pulsation history;
reading the arterial pulsation history;
determining, with the microcontroller, at least one limit including at least one of a pulse wave modulation value, a pulse rate value, and a blood flow value from the arterial pulsation history;
writing the at least one limit to a non-transitory computer readable medium;
receiving one or more new pulse data sets;
calculating at least one new variable value from the one or more new pulse data sets;
comparing, with the programmable hardware device, the at least one new variable value to the at least one limit; and
outputting a prompt from the microcontroller via a user interface to the person if a predetermined number of instances of the at least one new variable value falls outside the at least one limit.

2. The method for monitoring the hydration of a person of claim 1, further comprising:
determining and writing at least one new value of the at least one limit as a function of one or more instances of the one or more new pulse data sets.

3. The method for monitoring the hydration of a person of claim 2, wherein the at least one new value of the at least one limit is a function of at least a portion of at least one prior value of the at least one limit.

4. The method for monitoring the hydration of a person of claim 1, wherein comparing the at least one new variable value to the at least one limit further comprises:
comparing at least three successive instances of the at least one new variable value to the at least one limit; and
wherein outputting a prompt from the microcontroller via a user interface to the person if the predetermined number of instances of the at least one new variable value falls outside the at least one limit further comprises:
outputting the prompt if and only if each of the at least three successive instances of the at least one new variable value falls outside the at least one limit.

5. The method for monitoring the hydration of a person of claim 1, wherein measuring a physical periodic motion corresponding to a peripheral artery with a pulse sensor further comprises:
supporting one or more magnets on a flexible substrate adjacent to the person's skin such that the peripheral artery lies subjacent to the one or more magnets; and
detecting a magnetic field variation produced by the one or more magnets responsive to physical pulsations received from the subjacent peripheral artery with a magnetic sensor.

6. The method for monitoring the hydration of a person of claim 1, wherein determining, with the microcontroller at least one limit further comprises:
computing a standard deviation of instances of the arterial pulsation history; and
setting the at least one limit as two standard deviations greater than a mean arterial pulsation history value.

7. The method for monitoring the hydration of a person of claim 1, wherein determining, with the microcontroller, the limit further comprises:
computing a slope variable of a function of instances of the at least one value; and
setting the at least one limit as a derivative of the slope variable times a constant greater than one.

8. The method for monitoring the hydration of a person of claim 1, wherein the prompt indicates that the person is dehydrated.

9. The method for monitoring the hydration of a person of claim 8, further comprising outputting the prompt if the pulse rate falls outside the pulse rate limit and if the modulation falls outside the modulation limit.

10. The method for monitoring the hydration of a person of claim 1, wherein the modulation corresponds to a difference or ratio between systolic and diastolic peaks in expansion of the peripheral artery based on the magnetic sensor.

11. The method for monitoring the hydration of a person of claim 1, further comprising identifying the diastolic peak as a local maximum in expansion of the peripheral artery between a local minimum corresponding to a dicrotic notch and an overall minimum of a diameter of the peripheral artery as indicated by the magnetic sensor.

12. The method for monitoring the hydration of a person of claim 11, wherein the systolic peak corresponds to an overall maximum expansion of the peripheral artery as indicated by the magnetic sensor.

13. A non-transitory computer readable medium carrying computer executable instructions configured to cause a portable device to execute the method comprising the steps of:
measuring a physical periodic motion of a peripheral artery with a magnetic sensor of a health monitor sensor, each measured physical periodic motion including a modulation and a pulse rate;
receiving the modulation and pulse rate with a microcontroller and saving the modulation and pulse rate to a buffer memory as a modulation history and pulse rate history;
determining, with the microcontroller, a modulation, estimated instantaneous blood flow rate and pulse rate limit from the modulation and pulse rate history;
writing the modulation, blood flow and pulse rate limit to a non-transitory computer readable medium;
comparing, with the microcontroller, one or more measured instances of the modulation, blood flow and corresponding pulse rate to the modulation, blood flow and pulse rate limit; and
outputting a prompt from the microcontroller via a user interface to the person if the one or more measured instances of the modulation, blood flow and pulse rate falls outside the modulation and pulse rate limit.

14. The non-transitory computer readable medium carrying computer executable instructions configured to cause a portable device to execute the method of claim 13, wherein comparing the one or more measured instances of the modulation, blood flow and corresponding pulse rate to the modulation, blood flow and pulse rate limit further comprises:
comparing at least three successive measured instances to the combined modulation and pulse rate limit; and
wherein outputting a prompt from the microcontroller via a user interface to the person if the one or more measured instances of the modulation, blood flow and pulse rate falls outside the modulation, blood flow and pulse rate limit further comprises:
outputting the prompt if and only if each of the at least three successive measured instances falls outside the modulation, blood flow and pulse rate limit.

15. The non-transitory computer readable medium carrying computer executable instructions configured to cause a portable device to execute the method of claim 13, wherein measuring a physical periodic motion of a peripheral artery with a health monitor sensor further comprises:
supporting one or more magnets on a flexible substrate adjacent to the person's skin such that the peripheral artery lies subjacent to the one or more magnets; and
detecting a magnetic field variation produced by the one or more magnets responsive to physical pulsations received from the subjacent peripheral artery with a magnetic sensor.

16. The non-transitory computer readable medium carrying computer executable instructions configured to cause a portable device to execute the method of claim 13, wherein determining, with the microcontroller, the modulation and pulse rate limit from the modulation and pulse rate history further comprises:
computing a standard deviation variable of a function of instances of modulation and blood flow rate divided by corresponding pulse rate; and
setting the modulation and pulse rate limit as two standard deviations greater than a mean function value.

17. The non-transitory computer readable medium carrying computer executable instructions configured to cause a portable device to execute the method of claim 13, wherein determining, with the microcontroller, the modulation, blood flow and pulse rate limit from the modulation and pulse rate history further comprises:
computing a slope variable of a function of instances of modulation and blood flow rate divided by corresponding pulse rate; and
setting the modulation, blood flow and pulse rate limit as a derivative of the slope variable times a constant greater than one.

18. The non-transitory computer readable medium carrying computer executable instructions configured to cause a portable device to execute the method of claim 13, wherein the prompt indicates that the person is dehydrated.

19. The non-transitory computer readable medium carrying computer executable instructions configured to cause a portable device to execute the method of claim 18, further comprising outputting the prompt if the pulse rate falls outside the pulse rate limit and if the modulation falls outside the modulation limit.

20. The non-transitory computer readable medium carrying computer executable instructions configured to cause a portable device to execute the method of claim 13, wherein the modulation corresponds to a difference or ratio between systolic and diastolic peaks in expansion of the peripheral artery based on the magnetic sensor.

21. The non-transitory computer readable medium carrying computer executable instructions configured to cause a portable device to execute the method of claim 13, further comprising identifying the diastolic peak as a local maximum in expansion of the peripheral artery between a local minimum corresponding to a dicrotic notch and an overall minimum of a diameter of the peripheral artery as indicated by the magnetic sensor.

22. The non-transitory computer readable medium carrying computer executable instructions configured to cause a portable device to execute the method of claim 21, wherein the systolic peak corresponds to an overall maximum expansion of the peripheral artery as indicated by the magnetic sensor.

23. A method comprising:
generating, with a magnetic sensor positioned adjacent to a peripheral artery of a person, sensor signals indicative of movement of the peripheral artery;
calculating a pulse rate and a modulation of the peripheral artery based on the sensor signals;
comparing the pulse rate and the modulation to reference pulse rates and modulations;
determining a state of hydration of the person based on the comparison of the pulse rate and modulation to the reference pulse rates and modulation; and
outputting an alert to the user if the state of hydration corresponds to the person being dehydrated.

24. The method of claim 23, wherein the modulation corresponds to a difference between systolic and diastolic peaks in expansion of the peripheral artery as indicated by the sensor signals.

25. The method of claim 23, wherein the modulation corresponds to a ratio between systolic and diastolic peaks in expansion of the peripheral artery as indicated by the sensor signals.

26. The method of claim 23, further comprising determining that the person is dehydrated based on an increase in pulse rate compared to the reference pulse rate and a decrease in modulation compared to the reference modulation.

27. The method of claim 23, further comprising generating the reference pulse rate and modulation based on previous motion of the peripheral artery as indicated by sensor signals previously generated by the magnetic sensor.
